(19) United States
(12) Patent Application Publication
(10) Pub. No.: US 2023/0398371 A1
(43) Pub. Date: Dec. 14, 2023

(54) Intra-Oral Light Block for Performing Photo-Biomodulation

(71) Applicants: Ross Lefkowitz, Philadelphia, PA (US); Brian Pryor, Newark, DE (US); Sean Xiaolu Wang, Wilmington, DE (US)

(72) Inventors: Ross Lefkowitz, Philadelphia, PA (US); Brian Pryor, Newark, DE (US); Sean Xiaolu Wang, Wilmington, DE (US)

(73) Assignee: SBR Biotechnologies, Inc., Newark, DE (US)

(21) Appl. No.: 18/204,433

(22) Filed: Jun. 1, 2023

Related U.S. Application Data
(60) Provisional application No. 63/350,897, filed on Jun. 10, 2022.

(51) Int. Cl. A61N 5/06

(52) U.S. Cl. CPC A61N 5/0613; A61N 2005/0606; A61N 2005/0653

(57) ABSTRACT

The photo therapy apparatus of the present invention comprises one or more light sources which are integrated with a molar bite block for providing photobiomodulation therapy to the oral mucosa of a cancer patient suffering from (or at risk of) Oral Mucositis. The light sources can be either internal, or external to the bite block, and either lasers, light emitting diodes (LEDs), lamps, or any other types of light sources which produce therapeutic light in a desired wavelength range. The therapeutic light is delivered through the bite block to the oral mucosa. The delivered light produces a photochemical and photophysical process e.g. increase the blood flow and circulation. This photochemical and photophysical process helps to reduce the incidence and severity of oral mucositis in cancer patients and helps improve outcomes of cancer therapy.

### CROSS-REFERENCE TO RELATED APPLICATIONS

This application claims inventions which were disclosed in Provisional Patent Application No. 63/350,897, filed Jun. 10, 2022, entitled “INTRA-ORAL LIGHT BLOCK FOR PERFORMING PHOTO-BIOMODULATION”. The benefit under 35 USC § 119(e) of the above-mentioned United States Provisional Application is hereby claimed, and the aforementioned application is hereby incorporated herein by reference.

### FIELD OF THE INVENTION

This invention generally relates to a photobiomodulation therapy apparatus, and more specifically to a photo therapy apparatus for treating oral mucositis in cancer patients.

### BACKGROUND OF THE INVENTION

Oral Mucositis is a common side effect of cancer treatment (i.e. chemotherapy and radiation therapy). Oral Mucositis is the severe ulceration or the oral mucosa which is often debilitating, causing patients to stop eating, and can lead to severe complications and morbidity. Photobiomodulation (PBM) therapy (a therapeutic dose of light to the tissue) is considered effective for preventing and treating oral mucositis incidence and severity. Currently, only in office PBM treatments are available which require patients to travel to an office every single day to get this potentially lifesaving treatment. It is thus the goal of the present invention to provide a convenient photobiomodulation therapy apparatus for self-administered, home use photobiomodulation therapy for patients at risk of, or suffering from Oral Mucositis. The photobiomodulation therapy apparatus comprises one or more light sources that are integrated with a molar bite block, on which a patent will secure with their 1st, 2nd, and pre-molars. The emitting light will irradiate the oral mucosa (i.e. buccal mucosa, tongue, soft palate, etc.), which will reduce the incidence and the severity of oral mucositis, and providing better quality of live for cancer patients.

### SUMMARY OF THE INVENTION

The photobiomodulation therapy apparatus comprises one or more light sources that are integrated with a molar bite block for providing photobiomodulation therapy to the oral mucositis of cancer patients suffering from or at risk of Oral Mucositis. The light sources can be either internal, or external to the bite block, and either lasers, light emitting diodes (LEDs), lamps, or any other types of light sources which produce therapeutic light in a desired wavelength range. The bight block is inserted into the patient's mouth at the 1st, 2nd and pre-molar locations. The geometry of the bite block ensures the patient's oral cavity is open wide enough to provide enough “optical space” to allow the light to irradiate the target tissue effectively. The therapeutic light is delivered through the bite block to key oral mucosa, such as buccal mucosa (bilateral), tongue (dorsum), side of tongue (bilateral), tongue (ventral)/floor of mouth, hard palate, and soft palate. The delivered light produces a photochemical and photophysical process e.g. increase the blood flow and circulation. This photochemical and photophysical process helps to reduce the incidence and severity of oral mucositis in cancer patients and help improve outcomes of cancer therapy.

### BRIEF DESCRIPTION OF DRAWINGS

The accompanying figures, where like reference numerals refer to identical or functionally similar elements throughout the separate views and which together with the detailed description below are incorporated in and form part of the specification, serve to further illustrate various embodiments and to explain various principles and advantages all in accordance with the present invention.

FIG. 1 illustrates a first exemplary embodiment of the photo therapy apparatus for self-administered, home use photobiomodulation therapy for patients at risk of, or suffering from Oral Mucositis; and

FIG. 2 illustrates a second exemplary embodiment of the photo therapy apparatus for self-administered, home use photobiomodulation therapy for patients at risk of, or suffering from Oral Mucositis.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

### DETAILED DESCRIPTION OF THE INVENTION

Before describing in detail embodiments that are in accordance with the present invention, it should be observed that the embodiments reside primarily in combinations of method steps and apparatus components related to an intra-oral light block for performing photo-biomodulation. Accordingly, the apparatus components and method steps have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

In this document, relational terms such as first and second, top and bottom, and the like may be used solely to distinguish one entity or action from another entity or action without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

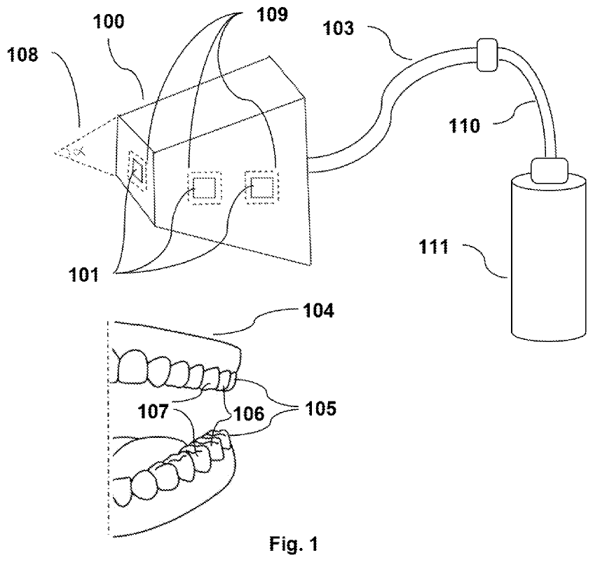

A first exemplary embodiment of the photo therapy apparatus is shown in FIG. 1. The photo therapy apparatus comprises one or more light sources 101 which are mounted on the sides off the bite block 100. The bite block 100 is a rigid or semi rigid geometry which a patient inserts into their mouth 104 at the 1^{st} molars 105, 2^{nd} molars 106, and pre-molars 107. The bite block 100 can either be a standard geometry or could be personalized to the patient via dental impressions (or other means). The angle 108 of the bite block 100 is designed to ensure the patients oral cavity is open wide enough to provide enough “optical space” to allow the light to irradiate the target tissue effectively. The proximal end of the bite block 100 is connected to an exit tube 103, which has a minimal cross section and has a curvature to exit the patient's mouth while limiting contact with soft tissue. In addition, this exit tube 103 is malleable which allows the patient to manipulate the geometry to avoid painful ulcers in the mouth, thus increasing patient comfort. The geometry of the bite block 100, light sources 101, and the exit tube 103, are designed to treat on both sides of the patient's mouth by revolving the apparatus 180 degrees about the exit tube 103 axis, and inserting it into the other side of their mouth 104 at the 1^{st} molars 105, 2^{nd} molars 106, and pre-molars 107. The light source 101 preferably consists of lasers such as laser diodes, diode pumped solid state lasers (DPSSLs), or light emitting diodes (LEDs), organic light emitting diode (OLED) sheets, and electrical luminance (EL) emitters, which produce therapeutic light in a desired wavelength range. The therapeutic light, which is delivered from the light sources 101 to the target tissue/oral mucosa and produces a variety of photochemical processes, e.g. increase of ATP (adenosine triphosphate), triggering of photo neurological response and activation of enzymes, changes in local pressure, increases in temperature, and deformation of cellular membranes. Such photochemical reactions help to reduce the incidence and severity of oral mucositis in cancer patients and help improve outcomes of cancer therapy. The light source 101 may also comprise lamp light sources or other types of broadband light sources such as supercontinuum light sources which are integrated with optical filters to select a desired output wavelength range. The light source 101 may be pulsed to enhance its treatment effect. The output power, duration, and pulsation property (i.e. pulse width, repetition rate, duty cycle) of the light source 101 is controlled by a controller 111 via wired connection 110 or wireless connection to adjust the light dosage applied to the breast tissue. Such properties of the light source 101 may be optimized in accordance to the patients individual mucosal and clinical parameters to produce the best therapeutic effect.

Depending on the output wavelength of the light source 101, the therapeutic light can provide a variety of treatment effects. The ultraviolet (UV)-blue light, at a wavelength of 370 to 490 nm, has high photon energy, which can help to produce singlet oxygen and effectively destroy bacteria. The red light, at a wavelength of 620 to 700 nm, can penetrate human tissue to a depth of about 8-10 mm. Skin layers, because of their high blood and water content, easily absorb red light, which helps to increase blood circulation and decrease inflammation/irritation. The near-infrared (NIR) light, at a wavelength of >700 nm, has been demonstrated to be beneficial for increasing cytochrome oxidase activity and ATP (adenosine triphosphate) content as well as promoting wound healing and relieving pain. In addition, the near-infrared light penetrates to a depth of about 30-40 mm, which makes it more effective for deep tissue. The light source 101 may comprise multiple light sources with different output wavelengths, each wavelength matching with the absorption band of a specific chromophore (water, hemoglobin, lipid, protein, etc.) of the subject oral mucosa.

In a slight variation of the present embodiment, an optical component 109 is inserted between front of the light source 101 in the bite block 101 for controlling the intensity, distribution, and angle of the therapeutic light. Such optical components can be reflective, refractive, diffractive, or transmissive elements, holographic diffuser, nanooptics, meta-optics, micro-lens array and digital optics for changing the direction and divergence of the light. As one example, the holographic diffuser or the micro-lens array may produce an unevenly distributed light intensity. This intensity modulation will help to further improve the therapeutic effect of the light source 101.

An additional variation of the present embodiment, a heat management component (not shown) is inserted behind the light source 101 in the bite block 101 to control overheating and prolong the treatment time as some of the treatment protocols may require. Such heat management components can be a heat sink at the light source 101, a thermal electric cooler, and convection (either mechanical or solid state). As one example, a fan can suck the hot air from inside the bite block 100 and exhaust out of the exit tube 103.

An additional variation of the present embodiment, an image capture system (not shown is integrated into the bite block 100 which can take images of a patient's oral mucosa.

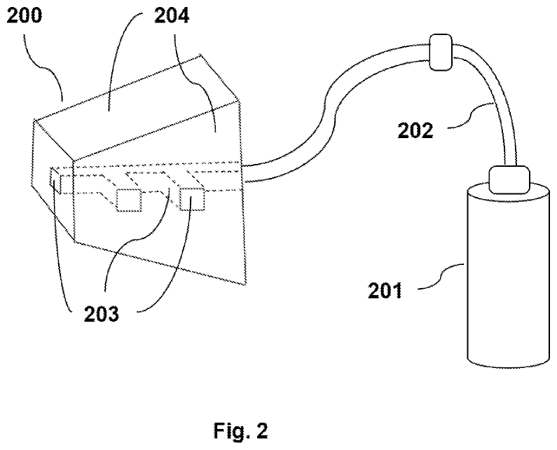

FIG. 2 illustrates a second exemplary embodiment of the photo therapy apparatus. Here the therapeutic light is produced by a light source 201 and delivered by an optical waveguide 202 into the bite block 200. Such optical waveguides 202 can be a limited single optical fiber, fiber bundle, or a liquid guide. The bite block 200 is made of a translucent material 203, and an opaque material 204. The translucent material serves as a side emitting light guide that scatters therapeutic light into the oral mucosa tissue to provide photo therapy treatment. The translucent material may comprise micro-sized particles and micro-V-grooves or other types of micro-structures for increasing the scattering of therapeutic light. The internal surface opaque material (not shown) is coated with an optically reflective material to minimize absorption and optical losses.

In the foregoing specification, specific embodiments of the present invention have been described. However, one of ordinary skill in the art appreciates that various modifications and changes can be made without departing from the scope of the present invention as set forth in the claims below. Accordingly, the specification and figures are to be regarded in an illustrative rather than a restrictive sense, and all such modifications are intended to be included within the scope of present invention. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

1. A photo therapy apparatus for treating oral mucosa in a cancer patient, the photobiomodulation therapy apparatus comprising:
a bite block for keeping the patient's mouth open at a predetermined position; and
a light source for producing therapeutic light;
wherein at least a section of the bite block is transparent to the therapeutic light and the therapeutic light is delivered through the bite block to treat the oral mucosa of the patient.

2. The photo therapy apparatus of claim 1, wherein the predetermined position is between a minimum position necessary to create enough optical space and a maximum position too wide to cause pain to the patient from oral mucositis.

3. The photo therapy apparatus of claim 1, wherein the light source is embedded inside the bite block.

4. The photo therapy apparatus of claim 1, wherein the therapeutic light is delivered through a light guide into the bite block.

5. The photo therapy apparatus of claim 1, further comprising an optical component for controlling an intensity distribution of the therapeutic light.

6. The photo therapy apparatus of claim 1, wherein the light source comprises lasers.

7. The photo therapy apparatus of claim 1, wherein the light source comprises light emitting diodes (LEDs).

8. The photo therapy apparatus of claim 1, wherein the light source comprises organic light emitting diodes (OLEDs).

9. The photo therapy apparatus of claim 1, wherein the light source comprises electrical luminance (EL) emitters.

10. The photo therapy apparatus of claim 1, wherein the light source comprises lamp light sources.

11. The photo therapy apparatus of claim 1, wherein the light source comprises supercontinuum light sources.

12. The photo therapy apparatus of claim 1, wherein the light source comprises optical filters for selecting a desired output wavelength range.
